(19) United States
(12) Patent Application Publication
(10) Pub. No.: US 2021/0137516 A1
(43) Pub. Date: May 13, 2021

(54) TROCAR-INTEGRATED SUTURING DEVICE

(71) Applicant: Mun Seok HAN, (US)

(72) Inventor: Mun Seok HAN, (US)

(21) Appl. No.: 16/968,598

(22) PCT Filed: May 15, 2020
(86) PCT No.: PCT/KR2020/006393

§ 371 Date: Aug. 9, 2020

(30) Foreign Application Priority Data
May 17, 2019 (KR) 1020180052178
Nov. 14, 2019 (KR) 1020190145531
Mar. 31, 2020 (KR) 1020200038782

(51) Int. Cl. A61B 17/04; A61B 17/34

(52) U.S. Cl. CPC A61B 17/0469; A61B 17/3423; A61B 2017/00004; A61B 17/0482; A61B 17/3403

(57) ABSTRACT

The present invention relates to a trocar-integrated suturing device that supplies a suture to a surgical site after laparoscopic surgery such that the end of the suture is supported by human tissues to easily make a knot when suturing the human tissues.

The trocar-integrated suturing device includes: a trocar including a cylindrical cannula being inserted into a human tissue and a body coupled to an upper portion of the cannula to be able to discharge gas existing in the human tissue; a needle ejector disposed to be movable up and down in the cannula and having a sharp punch at a front end to be able to penetrate flesh; a needle assembly supplying a suture into a human tissue using a needle moving in a pair of guide grooves disposed on an outer side of the cannula; and a head coupled to an upper portion of the body.

### TECHNICAL FIELD

The present invention relates to a trocar-integrated suturing device and, more particularly, to a trocar-integrated suturing device that supplies a suture to a portion, in which a trocar is inserted for a surgery, after laparoscopy, such that the end of the suture is supported by human tissues to easily make a knot when suturing the human tissues.

### BACKGROUND ART

When there is a problem with an organ in a human body, the organ is treated through laparotomy. However, laparotomy has a defect that there is a lot of hemorrhage and a large scar is left after the surgery due to a large cut.

Accordingly, surgical techniques that make small holes instead of laparotomy or use holes in a human body to treat internal organs are recently used. That is, new surgical techniques such as Minimal Invasive Surgery (MIS) and Natural Orifice Transluminal Endoscopic Surgery (NOTES) have been developed and used to overcome the defects of laparotomy.

MIS is a surgical technique that cuts minimal portions of the body of a patient using thin and long surgical instruments specifically designed to minimize cuts required for a surgery, and NOTES is a surgical technique that inserts surgical instruments through holes of the patient's body such as the anus or the gullet and moves the instruments to an object portion in the body of a patient.

MIS and NOTES have an advantage that the cuts for an operation are small, the amount of hemorrhage in an operation is considerably smaller than that of laparotomy, patients quickly recover after an operation, and scars that are exposed to the outside are small.

However, according to MIS (laparoscopic surgery using cannulas), since a cut (a portion through which a cannula is inserted) in a body is remarkably small in comparison to laparotomy, it is difficult and takes long time to suture the cut (a portion through which a cannula is inserted) in a general method.

In other words, according to the suturing method of laparoscopy in the related art, since a cut is directly sewn in a narrow space, expert techniques are required and it is highly difficult work, so suturing time increases and the operator also feels more fatigue.

Accordingly, the applicant(s) have registered Korean Patent No. 942060 by making an application of laparoscopic assistant instrument that is used to suture a surgical site in a human body in addition to maintaining the passages of surgical devices and instruments in laparoscopic surgery and includes a trocar having a body at the upper portion and a cannula at the lower portion, a suture deliverer having a pair of needles disposed with a predetermined gap and delivering a suture from a needle to the other needle, a guide disposed on the cannula to be able to slide up and down and having a suture guide groove at the lower portion, and a head coupled to the upper portion of the trocar in order to easily and quickly suture a surgical site in a human body by easily taking the end of a suture out of the human body after delivering the suture to the surgical site.

Meanwhile, several patent documents have been found out as the result of searching the prior art related to the present invention and some of them are introduced as follows.

Patent Document 1 has disclosed an organ suturing device including: a suturing needle that has a needle hole longitudinally formed on the center axis therein; a needle carrier that includes a needle fastener tube having a fastening tube hole formed on the center axis to be able to carry the suturing needle, thread-fastened to the rear end of the suturing needle, and having one or more openings on a portion of the outer surface; a suturing bead that is formed in a shape being able to pass through the needle hole and is connected with a suture; a bead pusher that has a pusher pin, which has a shape and a structure being able to push the suturing bead in the needle hole through the fastening tube hole to be able to carry the suturing bead through a hole in a needle, at the front end and has a pusher hole having a size formed on the center axis to be able to pass the suture; and a pusher handle that is coupled to the outer surface of the needle fastener tube and has a locking protrusion having a shape that can be locked to a pusher protrusion through an opening, whereby it is not required to separately knot a suture, it is possible to suture an organ at an exact depth even if the operator is not a highly skilled expert, and it is possible to reduce the suturing time.

Patent Document 2 has disclosed a surgical suturing device including a body, a suturing needle assembly mounted at a side of the body and accommodating at least one unit member composed of a pair of suturing needles and a suture connecting the pair of suturing needles, a needle push part disposed at the other side of the body and discharging the suturing needles out of the suturing needle assembly by applying pressure to the suturing needles, and a push adjuster connected to the needle push part and adjusting the position of the pressure selectively provided by the needle push part to the pair of suturing needles so that the pair of suturing needles is sequentially discharged, in which since the suturing needles are sequentially discharged, it is possible to reduce difficulty of an operation and increase convenience of an operation.

Patent document 3 has disclosed a laparoscopic suturing device including a holder and a cap that guide a suturing needle to a suturing position of a surgical site by moving through a hole of a cannula, a suture with a pre-formed knot, and a pusher that ejects the suture out of the holder, in which a suture connected with a suturing needle is knotted and kept in advance in a hole in the holder, the needle connected with a suture is temporarily stretched by connecting the cap to the front of the holder when the holder is inserted in the cannula, and the suture is ejected to a suturing portion from the holder by the pusher after the holder is inserted in an abdominal cavity, thereby making it simple and convenient to supply the suture to a surgical site in a body and knotting the suture after suturing.

### CITATION LIST

### Patent Literature

[Patent Literature 1]

(Patent Document 1) KR10-2006-0009698 A

(Patent Document 2) KR10-2011-0091054 A

(Patent Document 3) KR10-2016-0038385 A

(Patent Document 4) KR10-1942060 B1

### SUMMARY OF INVENTION

### Technical Problem

The present invention has been made in an effort to solve the problems, and an object of the present invention is to provide a trocar-integrated suturing device for suturing a human tissue that supplies a suture to a portion, in which a trocar is inserted for a surgery, after laparoscopic surgery, such that the end of the suture is supported by human tissues to easily make a knot when suturing the human tissues.

Another object of the present invention is to provide a trocar-integrated suturing device that makes it easy to suture tissues of a human body using a suture after an operation is finished by making it possible to quickly and accurately supply a suture into the tissues of a human body using a pair of needles having the front end of the suture therein.

### Solution to Problem

In order to achieve the objects, a trocar-integrated suturing device of the present invention includes: a trocar having a body at the upper portion and a cannula at the lower portion; a guide disposed to be movable up and down in the cannula and having a tapered front end; a suture support connected with a suture and moving out of the cannula through the front end of the guide; a cover blocking the tapered front end of the guide and forming a passage through which the suture support passes; a pusher disposed to be movable up and down in the guide and pushing the suture support; a punch disposed to be movable up and down at the lower portion of the cannula in order to be able to penetrate flesh, spaced a predetermined distance apart from the end of the guide, and having a hole passing the guide; and a head coupled to the upper portion of the trocar.

According to the trocar-integrated suturing device of the present invention, the cover is installed on the guide such that a hole passing the suture support is formed at a sharp end.

According to the trocar-integrated suturing device of the present invention, the cover has an upper end fixed and a lower end forming a door opening and closing a passage by rotating about a hinge disposed at a middle portion, and the door is formed in a size that can cover a lower end of the guide.

According to the trocar-integrated suturing device of the present invention, an upper end of the cover is fixed and a lower end of the cover opens and closes a passage by rotating about a recess formed in or at both sides of a middle portion of the cover, and a protrusion having a predetermined size is formed on an outer side of a lower end of the cover covering a lower end of the guide.

According to the trocar-integrated suturing device of the present invention, the suture support is made of a bioabsorbable material, and the suture is connected to a middle portion of the suture support.

A trocar-integrated suturing device of the present invention may include: a trocar including a cylindrical cannula being inserted into a human tissue and a body coupled to an upper portion of the cannula to be able to discharge gas existing in the human tissue; a needle ejector disposed to be movable up and down in the cannula and having a sharp punch at a front end to be able to penetrate flesh; a needle assembly supplying a suture into a human tissue using a needle moving in a pair of guide grooves disposed on an outer side of the cannula; and a head coupled to an upper portion of the body, supporting the needle ejector, and restricting movement of the needle assembly.

A trocar-integrated suturing device of the present invention may include: a trocar including a cylindrical cannula being inserted into a human tissue and a body coupled to an upper portion of the cannula to be able to discharge gas existing in the human tissue; a needle ejector disposed to be movable up and down in the cannula and having a sharp front end to be able to penetrate flesh; a needle assembly supplying a suture into a human tissue using a needle moving in a pair of guide grooves disposed on an outer side of the cannula; and a head coupled to an upper portion of the body, supporting the needle ejector, and restricting movement of the needle assembly.

According to the trocar-integrated suturing device of the present invention, the needle ejector includes: a needle ejection body having an enlarged portion at the front end at which an accommodation groove corresponding to the guide groove of the cannula is formed, and a reduced portion at the rear end on the outer side; a punch having a sharp front end to be able to penetrate the human tissue and having a guide groove for guiding the needle on the outer side; and a metal shaft connecting the needle ejection body and the punch.

According to the trocar-integrated suturing device of the present invention, the needle assembly has: a pair of needles having an accommodation groove, in which the end of the suture is accommodated, at the front end and moving up and down in the guide grooves of the cannula; and a handle formed on the needles by insert injection to move up and down the needle outside the head.

According to the trocar-integrated suturing device of the present invention, the head includes a main body coupled to the upper portion of the body, a push button disposed on a side of the main body to be able to decouple the body and the main body, and a rotary stopper rotatably coupled to the main body to restrict or allow movement of the needle assembly.

According to the trocar-integrated suturing device of the present invention, the suture has a barb on a portion of an end to be fixed in a human tissue.

### Advantageous Effects of Invention

According to the first embodiment of a trocar-integrated suturing device of the present invention, since a guide having a tapered front end is inserted into a cannula of a trocar, a suture and a suture support are pushed into a human tissue through the guide, and then the suture support is brought in close contact with the human tissue to support the suture, it is possible to suture a bored portion or a surgical site in a laparoscopic surgery, etc., outside, so there is an effect that convenience is considerably improved.

Further, according to the first embodiment of a trocar-integrated suturing device of the present invention, since a hole is formed or a door is provided at the sharp end of the guide, there is an effect that the suture support comes out of the guide and the cannula through the hole or the door and comes in close contact with a human tissue when the suture support is pushed by a pusher.

Further, according to the first embodiment of a trocar-integrated suturing device of the present invention, since a recess is formed in or at both sides of the middle portion of a cover and a protrusion is formed on the outer side of the lower end of the cover, there is an effect that it is possible to rotate the lower portion of the cover even without separately installing a hinge and it is also possible to prevent the cover from being stuck to the guide member.

Further, according to the first embodiment of a trocar-integrated suturing device of the present invention, since the suture support is made of a bioabsorbable material, the suture support is melt up as time passes, so it is not required to separately remove the suture support. Further, since the suture is connected to the middle portion of the suture support, the suture support is brought parallel in close contact with a human tissue, so there is an effect that the suture support stably supports the suture.

Further, according to a second embodiment and a third embodiment of a trocar-integrated suturing device of the present invention, since a suture is supplied into a human body by a needle inserted in a guide groove formed on the outer side of the cannula, there is an effect that it is easy to supply a suture.

Further, according to the second embodiment of a trocar-integrated suturing device of the present invention, since the punch of a needle ejector and a needle ejection body are connected by a shaft and spaced apart from each other, a needle having a suture penetrates a human tissue positioned between the punch and the needle ejection body, whereby it is easy to supply a suture into a human tissue and it is also easy to suture a hole of a human body formed by the cannula.

Further, according to the second embodiment and the third embodiment of a trocar-integrated suturing device of the present invention, since a pair of needles is integrated with a handle by insert injection, there is an effect that it is possible to easily move the needle using the handle and it is easy to supply a suture through the needles.

Further, according to the second embodiment and the third embodiment of a trocar-integrated suturing device of the present invention, since a rotary stopper is disposed on the head and restricts movement of the needle assembly, there is an effect that damage due to unexpected movement of the needles is prevented.

Further, according to the second embodiment and the third embodiment of a trocar-integrated suturing device of the present invention, since a barb is formed at the end of the suture, there is an effect that the suture supplied in a human tissue is fixed without being pulled out.

### BRIEF DESCRIPTION OF DRAWINGS

FIG. 1 is a perspective view showing the external appearance of a first embodiment of a trocar-integrated suturing device according to the present invention.

FIG. 2 is a reference view showing the state in which a suture is fixed at a surgical site using the first embodiment of a trocar-integrated suturing device according to the present invention.

FIG. 3 is a cross-sectional view showing the inside of a guide of the trocar-integrated suturing device according to the present invention.

FIG. 4 is a cross-sectional view showing the structure of a guide and a cover according to the present invention.

FIG. 5 is a reference view showing another embodiment of the cover according to the present invention.

FIG. 6 is a perspective view showing the external appearance of a second embodiment of a trocar-integrated suturing device according to the present invention.

FIG. 7 is an exploded perspective view showing the second embodiment of a trocar-integrated suturing device according to the present invention.

FIG. 8 is an enlarged view showing the front end portion of a needle ejector of the second embodiment of a trocar-integrated suturing device according to the present invention.

FIG. 9 is a view showing the configuration of a needle assembly of the second embodiment of a trocar-integrated suturing device according to the present invention.

FIG. 10 is a view showing a body of the second embodiment of a trocar-integrated suturing device according to the present invention.

FIG. 11 is an enlarged view showing the end portion of a suture that is applied to the second embodiment of a trocar-integrated suturing device according to the present invention.

FIG. 12 is a reference view showing a process of supplying a suture using the second embodiment of a trocar-integrated suturing device according to the present invention.

FIG. 13 is a perspective view showing the external appearance of a third embodiment of a trocar-integrated suturing device according to the present invention.

FIG. 14 is an exploded perspective view showing the third embodiment of a trocar-integrated suturing device according to the present invention.

FIG. 15 is an enlarged view showing the front end portion of a needle ejector of the third embodiment of a trocar-integrated suturing device according to the present invention.

FIG. 16 is a reference view showing a process of supplying a suture using the trocar-integrated suturing device according to the present invention.

### DESCRIPTION OF EMBODIMENTS

Hereinafter, a trocar-integrated suturing device according to the present invention will be described with reference to the accompanying drawings.

In order to describe preferred embodiments of the present invention, FIG. 1 is a perspective view showing the external appearance of a first embodiment of a trocar-integrated suturing device according to the present invention, FIG. 2 is a reference view showing the state in which a suture is fixed at a surgical site using the first embodiment of a trocar-integrated suturing device according to the present invention, FIG. 3 is a cross-sectional view showing the inside of a guide of the trocar-integrated suturing device according to the present invention, FIG. 4 is a cross-sectional view showing the structure of a guide and a cover according to the present invention, and FIG. 5 is a reference view showing another embodiment of the cover according to the present invention.

A first embodiment of a trocar-integrated suturing device according to the present invention, as shown in FIGS. 1 to 5, includes: a trocar 10 having a body at the upper portion and a cannula 12 at the lower portion; a guide 20 disposed to be movable up and down in the cannula 12 and having a tapered front end; a suture support 30 connected with a suture 35 and moving out of the cannula 12 through the front end of the guide 20; a cover 40 blocking the tapered front end of the guide 20 and forming a passage through which the suture support 30 passes; a pusher 50 disposed to be movable up and down in the guide 20 and pushing the suture support 30; a punch disposed to be movable up and down at the lower portion of the cannula 12 in order to be able to penetrate flesh, spaced a predetermined distance apart from the end of the guide 20, and having a hole 65 passing the guide 20; and a head 70 coupled to the upper portion of the trocar 10.

It is preferable that the suture 35 is made of a bioabsorbable material, that the suture support 30 is also made of a bioabsorbable material, and that the suture 35 is connected to the middle portion of the suture support 30. The suture support 30 may be formed by overlapping and then melting or bonding the end of a suture with a bioabsorbable material for integration. The reason that the suture 35 and the suture support 30 are made of a bioabsorbable material, as described above, is to enable the suture support 30 to be absorbed into human tissues without acting as a foreign substance while having strength at a predetermined level or higher. Further, the reason that the suture 35 is connected to the middle portion of the suture support 30 is for bringing the suture support 30 parallel in close contact with a human body 100, as shown in FIG. 2, when the suture 35 is pulled after the suture support 30 is inserted into a human tissue.

Accordingly, an operator forms a hole in the human tissue 100 using the punch 60, inserts a surgical instrument (not shown) with the body 11 and the cannula 12 of the trocar 10, performs laparoscopic surgery, and then lifts the lower portion of the human body with the hole using the punch 60 with the surgical instruments separated from the cannula 12, thereby pushing the human tissue into the hole, as shown in FIG. 2. In this state, the operator bores the human body using the sharp end of the guide 20 by pushing the guide 20, passes the end through the hole 65 of the punch 60, and then inserts the suture 35 and the suture support 30 into the human body 100 through the guide 20. That is, the suture support 30 is pushed to the lower portion of the human tissue 100 by pushing the suture support 30 at the end in the guide 20 with the pusher 50. Thereafter, the operator moves down the punch 60 such that the guide 20 is positioned between the cannula 12 and the punch 60, moves up the guide 20 with the pusher 50, and then pulls the suture 35 such that the suture support 30 supports the suture 35 in close contact with the human tissue 100.

The cover 40 is disposed at the front end of the guide 20 such that the suture support 30 can be moved out of the guide 20 when the suture support 30 is pushed by the pusher 50, and as shown in FIG. 3, a hole 41 passing the suture support 30 is formed at the sharp end. However, considering that the diameter of the guide 20 is not more than about 1 to 2 mm, it is difficult to form the hole 41 that can pass the suture support 30 at the end of the guide 20.

Accordingly, it is preferable to use a portion of the cover 40 as a door 43 such that the suture support 30 is easily moved out of the guide 20 when the suture support 30 is pushed by the pusher 50. That is, the upper end of the cover 40 is fixed, and the lower end of the thereof is used as a door 43. In detail, as shown in (a) of FIG. 5, a hinge 42 is disposed at the middle portion between the upper portion and the lower portion of the cover 40 such that the door 43 rotates about the hinge 42 to open/close a passage. However, in this case, it is further preferable that the door 43 is formed in a size that can cover the lower end of the guide 20 so that the door 43 can freely rotate without being stuck to the guide 20.

However, considering the cover 40 of which the size depends on the guide 20, it is difficult to install the hinge 42 in the cover 40, so it is further preferable to enable a door to rotate and open/close the passage without installing the hinge 42.

That is, as shown in (b) and (c) of FIG. 5, the upper end of the cover 40 is fixed to the guide 20 and a recess 44 is formed in or at both sides of the middle portion such that the lower portion of the cover 40 opens/closes the passage by rotating about the recess 44. A protrusion 45 having a predetermined size is formed at the lower end of the cover 40 that covers the lower end of the guide 20 so that the passage can be easily opened even by a small force and the end of the cover 40 is not stuck to the guide 20.

Meanwhile, in order to describe other embodiments of a trocar-integrated suturing device according to the present invention, FIG. 6 is a perspective view showing the external appearance of a second embodiment of a trocar-integrated suturing device according to the present invention, FIG. 7 is an exploded perspective view showing the second embodiment of a trocar-integrated suturing device according to the present invention, and FIG. 8 is an enlarged view showing the front end portion of a needle ejector of the second embodiment of a trocar-integrated suturing device according to the present invention. FIG. 9 is a view showing the configuration of a needle assembly of the second embodiment of a trocar-integrated suturing device according to the present invention, FIG. 10 is a view showing a body of the second embodiment of a trocar-integrated suturing device according to the present invention, FIG. 11 is an enlarged view showing the end portion of a suture that is applied to the second embodiment of a trocar-integrated suturing device according to the present invention, and FIG. 12 is a reference view showing a process of supplying a suture using the second embodiment of a trocar-integrated suturing device according to the present invention. FIG. 13 is a perspective view showing the external appearance of a third embodiment of a trocar-integrated suturing device according to the present invention, FIG. 14 is an exploded perspective view showing the third embodiment of a trocar-integrated suturing device according to the present invention, FIG. 15 is an enlarged view showing the front end portion of a needle ejector of the third embodiment of a trocar-integrated suturing device according to the present invention, and FIG. 16 is a reference view showing a process of supplying a suture using the trocar-integrated suturing device according to the present invention.

A second embodiment of a trocar-integrated suturing device according to the present invention, as shown in FIGS. 6 to 11, includes a body 110, a cannula 120, a head 130, a needle ejector 140, and a needle assembly 150.

The body is a part in which surgical instruments are operated in laparoscopic surgery, etc., can discharge gas existing in a human tissue 200, and is coupled to the upper portion of the cannula 120. To this end, the body 110 includes a cannular body 111 coupled to the upper portion of the cannula 120, a cannular cap 112 coupled to the upper portion of the cannular body 111, a gas tube 113 disposed at a side of the cannular body 111 to discharge gas in the human tissue 200, and a valve 114 disposed at the upper portion of the gas tube 113 so that discharge of gas can be adjusted. It is preferable that the body 100 further includes: a first silicon tube 115 formed in a conical shape such that the needle ejector 140 is inserted therein without gas being discharged, and being in close contact with the needle ejector 140; an upper inner pipe 117 and a lower inner pipe 118 disposed in the cannular cap 112 to support the needle ejector 140; and a second silicon tube 116 disposed between the cannular cap 112 and the inner pipes 117 and 118 and pressing the inner pipes 117 and 118 toward the center.

The cannula 120, which is a part designed to be inserted into the human tissue 200, is formed in a cylindrical shape to form a passage through which surgical instruments, etc. are inserted. A guide groove 125 for guiding a needle 151 of the needle assembly 150 is longitudinally formed on the outer side of the cannula 120. The guide groove 125 is recessed toward the center at the outer side of the cannula 120.

The head 130 is coupled to the upper portion of the body 110, thereby supporting the needle ejector 140 and restricting movement of the needle assembly 150. To this end, the head 130 includes a main body 131 coupled to the upper portion of the body 110, a push button 133 disposed on a side of the main body 131 to be able to decouple the body 110 and the main body 131, and a rotary stopper 135 rotatably coupled to the main body 131 to restrict or allow movement of the needle assembly 150. Accordingly, the needle assembly 150 can be moved up and down only when the rotary stopper 135 is rotated at a predetermined degree, and the needle assembly 150 is fixed without moving when the rotary stopper 135 is returned to the initial position.

The needle ejector 140, which is a part for penetrating flesh using a punch 145 at the front end, is disposed to be movable up and down in the cannula 120. In detail, the needle ejector 140, as shown in FIGS. 7 and 8, includes: a needle ejection body 141 having an enlarged portion at the front end at which an accommodation groove 142 corresponding to the guide groove 125 of the cannula 120 is formed, and a reduced portion at the rear end on the outer side; a punch 145 having a pointed front end to be able to penetrate the human tissue 200 and having a guide groove 146 for guiding the needle 151 on the outer side; and a metal shaft 143 connecting the needle ejection body 141 and the punch 145. Here, it is preferable to form the front end of the needle ejection body 141 in a conical shape corresponding to the punch 145 such that when the cannula 120 is moved forward, the needle ejection body 141 moves forward while expanding a hole in the human tissue 200 in order that the cannula 120 can be smoothly moved. It is further preferable that the shaft 143 is made of stainless steel that is not rusted, and the needle ejection body 141 and the punch 145 are made of plastic.

The needle assembly 150, which is a part for supplying a suture 160 into the human tissue 200, has a needle 151 moving in a pair of guide groove 125 formed on the outer side of the cannula 120. In detail, the needle assembly 150, as shown in FIG. 9, includes: a pair of needles 151 having an accommodation groove 153, in which the end of the suture 160 is accommodated, at the front end, and moving up and down in the guide grooves 125 of the cannula 120; and a handle 155 for moving up/down the needle 151 outside the head 130. Here, it is preferable that the needle 151 and the handle 155 are integrated by insert injection.

It is preferable that the suture 160 has one or more barbs 165 at a portion of the end to be fixed to the human tissue 200 by the barbs 165. When the suture 160 is moved forward, the barbs 165 formed at the end of the suture 160 is reclined and does not restrict the movement, but when the suture 160 is moved backward, the barbs 165 are erected and get stuck in the human tissue 200, thereby preventing the suture 160 from being pulled out. If there are no barbs 165 on the suture 160, it may be possible to make the end of the suture 160 rough to prevent the suture 160 supplied in the human tissue 20 from passing through the human tissue 200.

Supplying a suture into a surgical site using the second embodiment of a trocar-integrated suturing device having the configuration described above of the present invention is described hereafter with reference to FIG. 11.

An operator inserts the needle ejector 140 into the cannula 120 coupled to the body 110 and inserts the needle 151 of the needle assembly 150 through the head 130 and the body 110. In this case, the end of the suture 160 should be accommodated in the accommodation groove 153 of the needle 151 and positioned in the guide grooves 125 formed on the outer side of the cannula 120. The needle assembly 150 is inserted such that the end of the needle 151 is positioned at a predetermined distance from the end of the cannula 120. In this state, the operator thrusts the needle ejector 140 into the human tissue 200 such that the punch 145 of the needle ejector 140 completely penetrates the human tissue to be inserted into the human tissue 200. Accordingly, the human tissue 200 is naturally restored and positioned between the punch 145 of the needle ejector 140 and the front end of the needle ejection body 141.

In this state, when the rotary stopper 135 of the head 130 is rotated 45 degrees, the needle assembly 150 can be moved. Accordingly, when an operator presses the handle 155 of the needle assembly 150 such that the needle 151 can be moved forward, the front end of the needle 151 penetrates the human tissue 200 to be inserted into the human tissue 200. When the needle 151 completely penetrates the human tissue 200, the end of the suture 160 accommodated in the accommodation groove 153 of the needle 151 comes outside and is positioned in the human tissue 200.

Thereafter, the operator moves back the needle assembly 150 by pulling the handle 155 and returns the rotary stopper 133 to the initial position, thereby fixing the needle assembly 150. In this process, the needle 151 made of metal in a cylindrical shape is smoothly moved back, but the suture 160 is not moved back with the needle 151 and fixed in the human tissue 200 by the barbs 165 at the end.

When the suture 160 is fixed through this process, the operator moves forward the trocar 100 composed of the cannula 120 and the body 110 such that the cannula 120 penetrates the human tissue 200, thereby securing a passage through which a surgical instrument, etc. can be inserted. That is, when the cannula 120 completely penetrates the human tissue 200, the operator separates the head 130 from the body 110 using the push button 133 of the head 130 and moves back all of the head 130, the needle ejector 140, and the needle assembly 150 such that only the cannula 120 and the body 120 penetrating the human tissue 200, and the suture 160 are left in the human tissue 200. In this state, the inside of the cannular cap 112 of the body 110 is blocked by elastic force of the first silicon tube 115 and the second silicon tube 116 of the body 110, so external air cannot flow into the human tissue 200, whereby infection due to external air is prevented.

As described above, when a passage is formed by the cannula 120, it is possible to perform laparoscopic surgery or surgery by inserting surgical instruments, etc. into the human tissue 200 through the body 110 and the cannula 120. Further, an operator takes out the surgical instruments, etc. after an operation or surgery, separates the cannula 120 from the human tissue 200, and then sutures the surgical site using the suture 160 exposed outside the human tissue 200.

Although the needle ejection body 141 and the punch 145 of the needle ejector 140 are connected by the metal shaft 143 in the description of the second embodiment, it may be possible to make the front end of the needle ejector 141 sharp and not use the punch 145.

In detail, a third embodiment of a trocar-integrated suturing device according to the present invention, as shown in FIGS. 13 to 15, includes the body 110, the cannula 120, the head 130, the needle ejector 140, and the needle assembly 150, and the body 110, the cannula 120, the head 130, and the needle assembly 150 are the same as those of the second embodiment, description thereof is omitted.

However, the needle ejector 140 includes a needle ejection body 141 having an accommodation groove 142 corresponding to the guide grooves 125 of the cannula 120 on the outer side and having a sharp front end to be able to penetrate the human tissue 200. The reason of forming the front end of the needle ejector body 141 in a sharp conical shape is for smoothly moving forward the needle ejector body 141 while expanding a hole in the human tissue 200 in order that the cannula 120 can be smoothly moved when the cannula 120 is moved forward. It is preferable that the needle ejection body 141 is made of plastic.

Supplying a suture into a surgical site using the third embodiment of a trocar-integrated suturing device of the present invention is described hereafter with reference to FIG. 16.

An operator inserts the needle ejector 140 into the cannula 120 coupled to the body 110 and inserts the needle 151 of the needle assembly 150 through the head 130 and the body 110. In this case, the end of the suture 160 should be accommodated in the accommodation groove 153 of the needle 151 and positioned in the guide grooves 125 formed on the outer side of the cannula 120. The needle assembly 150 is inserted such that the end of the needle 151 is positioned at a predetermined distance from the end of the cannula 120. In this state, the operator thrusts the needle ejector 140 and the cannula 120 into the human tissue 200 such that the front end of the needle ejector 140 completely penetrates the human tissue 200 to be inserted into the human tissue 200. Accordingly, when the cannula 120 is moved back, the human tissue 200 is naturally restored and positioned between the needle ejector 140 and the cannula 120.

In this state, when the rotary stopper 135 of the head 130 is rotated 45 degrees, the needle assembly 150 can be moved. Accordingly, when an operator presses the handle 155 of the needle assembly 150 such that the needle 151 can be moved forward, the front end of the needle 151 penetrates the human tissue 200 to be inserted into the human tissue 200. When the needle 151 completely penetrates the human tissue 200, the end of the suture 160 accommodated in the accommodation groove 153 of the needle 151 comes outside and is positioned in the human tissue 200.

Thereafter, the operator moves back the needle assembly 150 by pulling the handle 155 and returns the rotary stopper 133 to the initial position, thereby fixing the needle assembly 150. In this process, the needle 151 made of metal in a cylindrical shape is smoothly moved back, but the suture 160 is not moved back with the needle 151 and fixed in the human tissue 200 by the barbs 165 at the end.

When the suture 160 is fixed through this process, the operator moves forward the trocar 100 composed of the cannula 120 and the body 110 such that the cannula 120 penetrates the human tissue 200, thereby securing a passage through which a surgical instrument, etc. can be inserted. That is, when the cannula 120 completely penetrates the human tissue 200, the operator separates the head 130 from the body 110 using the push button 133 of the head 130 and moves back all of the head 130 the needle ejector 140, and the needle assembly 150 such that only the cannula 120 and the body 120penetrating the human tissue 200, and the suture 160 are left in the human tissue 200. In this state, the inside of the cannular cap 112 of the body 110 is blocked by elastic force of the first silicon tube 115 and the second silicon tube 116 of the body 110, so external air cannot flow into the human tissue 200, thereby preventing infection due to external air.

As described above, when a passage is formed by the cannula 120, it is possible to perform laparoscopic surgery or surgery by inserting surgical instruments, etc. into the human tissue 200 through the body 110 and the cannula 120. Further, an operator takes out the surgical instruments, etc. after an operation or surgery, separates the cannula 120 from the human tissue 200, and then sutures the surgical site using the suture 160 exposed outside the human tissue 200.

Although some embodiments were described above to exemplify the spirit of the present invention, the present invention is not limited only to the configuration and operation described above, and it will be understood by those skilled in the art that the present invention may be changed and modified in various ways without departing from the spirit of present invention described herein and claims. Therefore, all the appropriate changes, modifications, and equivalents should be considered as being included in the range of the present invention.

1. A trocar-integrated suturing device comprising:
a trocar having a body at the upper portion and a cannula at the lower portion;
a guide disposed to be movable up and down in the cannula and having a tapered front end;
a suture support connected with a suture and moving out of the cannula through the front end of the guide;
a cover blocking the tapered front end of the guide member and forming a passage through which the suture support passes;
a pusher disposed to be movable up and down in the guide and pushing the suture support;
a punch disposed to be movable up and down at the lower portion of the cannula in order to be able to penetrate flesh, spaced a predetermined distance apart from the end of the guide, and having a hole passing the guide; and
a head coupled to the upper portion of the trocar.

2. The trocar-integrated suturing device of claim 1, wherein the cover is installed on the guide such that a hole passing the suture support is formed at a sharp end.

3. The trocar-integrated suturing device of claim 1, wherein the cover has an upper end fixed and a lower end forming a door opening and closing a passage by rotating about a hinge disposed at a middle portion, and
the door is formed in a size that can cover a lower end of the guide.

4. The trocar-integrated suturing device of claim 1, wherein an upper end of the cover is fixed and a lower end of the cover opens and closes a passage by rotating about a recess formed in or at both sides of a middle portion of the cover, and a protrusion having a predetermined size is formed on an outer side of a lower end of the cover covering a lower end of the guide.

5. The trocar-integrated suturing device of claim 1, wherein the suture support is made of a bioabsorbable material and the suture is connected to a middle portion of the suture support.

6. A trocar-integrated suturing device comprising:
a trocar including a cylindrical cannula being inserted into a human tissue and a body coupled to an upper portion of the cannula to be able to discharge gas existing in the human tissue;
a needle ejector disposed to be movable up and down in the cannula and having a sharp punch at a front end to be able to penetrate flesh;
a needle assembly supplying a suture into a human tissue using a needle moving in a pair of guide grooves disposed on an outer side of the cannula; and
a head coupled to an upper portion of the body, supporting the needle ejector, and restricting movement of the needle assembly.

7. The trocar-integrated suturing device of claim 6, wherein the needle ejector includes: a needle ejection body having an enlarged portion at the front end at which an accommodation groove corresponding to the guide groove of the cannula is formed, and a reduced portion at the rear end on the outer side; a punch having a pointed front end to be able to penetrate the human tissue and having a guide groove for guiding the needle on the outer side; and a metal shaft connecting the needle ejection body and the punch.

8. A trocar-integrated suturing device comprising:
a trocar including a cylindrical cannula being inserted into a human tissue and a body coupled to an upper portion of the cannula to be able to discharge gas existing in the human tissue;
a needle ejector disposed to be movable up and down in the cannula and having a sharp front end to be able to penetrate flesh;
a needle assembly supplying a suture into a human tissue using a needle moving in a pair of guide grooves disposed on an outer side of the cannula; and
a head coupled to an upper portion of the body, supporting the needle ejector, and restricting movement of the needle assembly.

9. The trocar-integrated suturing device of claim 6, wherein the needle assembly has: a pair of needles having an accommodation groove, in which the end of the suture is accommodated, at the front end, and moving up and down in the guide grooves of the cannula; and a handle formed on the needles by insert injection to move up and down the needle outside the head.

10. The trocar-integrated suturing device of claim 8, wherein the needle assembly has: a pair of needles having an accommodation groove, in which the end of the suture is accommodated, at the front end, and moving up and down in the guide grooves of the cannula; and a handle formed on the needles by insert injection to move up and down the needle outside the head.

11. The trocar-integrated suturing device of claim 6, wherein the head includes a main body coupled to the upper portion of the body, a push button disposed on a side of the main body to be able to decouple the body and the main body, and a rotary stopper rotatably coupled to the main body to restrict or allow movement of the needle assembly.

12. The trocar-integrated suturing device of claim 8, wherein the head includes a main body coupled to the upper portion of the body, a push button disposed on a side of the main body to be able to decouple the body and the main body, and a rotary stopper rotatably coupled to the main body to restrict or allow movement of the needle assembly.

13. The trocar-integrated suturing device of claim 6, wherein the suture has a barb on a portion of an end to be fixed in a human tissue.

14. The trocar-integrated suturing device of claim 8, wherein the suture has a barb on a portion of an end to be fixed in a human tissue.
